(19) United States
(12) Patent Application Publication
(10) Pub. No.: US 2023/0326580 A1
(43) Pub. Date: Oct. 12, 2023

(54) INFORMATION PROCESSING APPARATUS, INFORMATION PROCESSING METHOD, AND INFORMATION PROCESSING PROGRAM

(71) Applicant: FUJIFILM Corporation, Tokyo (JP)

(72) Inventor: Takuya YUZAWA, Tokyo (JP)

(73) Assignee: FUJIFILM Corporation, Tokyo (JP)

(21) Appl. No.: 18/186,949

(22) Filed: Mar. 21, 2023

(30) Foreign Application Priority Data
Mar. 25, 2022 (JP) 2022-050806

(51) Int. Cl. G06T 7/00; G16H 30/40; G06T 7/60

(52) U.S. Cl. CPC G16H 30/40; G06T 7/0016; G06T 7/60; G06T 2207/30004; G06T 2207/30176

(57) ABSTRACT

An information processing apparatus comprising at least one processor, wherein the at least one processor is configured to: acquire a first measurement value measured for a first region of interest included in a first image obtained by imaging a subject at a first point in time; acquire a second document describing diagnosis content based on a second image obtained by imaging the subject at a second point in time; determine whether or not the second document includes a second measurement value of the same type as the first measurement value, which is measured for a second region of interest included in the second image, the second region of interest being the same as the first region of interest; and in a case where the determination is a negative determination, acquire the second measurement value.

### CROSS-REFERENCE TO RELATED APPLICATIONS

This application claims priority from Japanese Application No. 2022-050806, filed on Mar. 25, 2022, the entire disclosure of which is incorporated herein by reference.

### BACKGROUND

### Technical Field

The present disclosure relates to an information processing apparatus, an information processing method, and an information processing program.

### Related Art

In the related art, image diagnosis is performed using medical images obtained by imaging apparatuses such as computed tomography (CT) apparatuses and magnetic resonance imaging (MRI) apparatuses. In addition, medical images are analyzed via computer aided detection/diagnosis (CAD) using a discriminator in which learning is performed by deep learning or the like, and regions of interest including structures, lesions, and the like included in the medical images are detected and/or diagnosed. The medical images and analysis results via CAD are transmitted to a terminal of a healthcare professional such as a radiologist who interprets the medical images. The healthcare professional such as a radiologist interprets the medical image by referring to the medical image and analysis result using his or her own terminal and creates an interpretation report.

In addition, various methods have been proposed to support the creation of interpretation reports in order to reduce the burden of the interpretation work. For example, JP2019-153250A discloses a technique for creating an interpretation report based on a keyword input by a radiologist and an analysis result of a medical image. In the technique described in JP2019-153250A, a sentence to be included in the interpretation report is created by using a recurrent neural network trained to generate a sentence from input characters.

Further, for example, in regular health checkups and post-treatment follow-up observations, the same subject may be examined a plurality of times and a change over time in a medical condition may be confirmed by performing comparative interpretation of medical images at each point in time. Therefore, various methods for performing comparative interpretation have been proposed. For example, WO2018/159363A discloses performing a comparison process between first diagnosis time-specific identification information acquired at the time of a first diagnosis and second diagnosis time-specific identification information acquired at the time of a second diagnosis different from that at the time of the first diagnosis.

Incidentally, even though an analysis result of a medical image is obtained via CAD, the analysis result may not actually be described in the interpretation report, depending on the determination of a creator of the interpretation report. On the other hand, a lesion that was once determined not to be described in the interpretation report may be determined to be required to be described at the time of the re-examination due to deterioration over time or a change in the creator. In order to perform comparative interpretation in such a case, it takes time and effort to re-obtain the analysis result at a point in time not described in the interpretation report.

### SUMMARY

The present disclosure provides an information processing apparatus, an information processing method, and an information processing program capable of supporting comparative interpretation.

According to a first aspect of the present disclosure, there is provided an information processing apparatus comprising at least one processor, in which the processor is configured to: acquire a first measurement value measured for a first region of interest included in a first image obtained by imaging a subject at a first point in time; acquire a second document describing diagnosis content based on a second image obtained by imaging the subject at a second point in time; determine whether or not the second document includes a second measurement value of the same type as the first measurement value, which is measured for a second region of interest that is included in the second image and is the same as the first region of interest; and in a case where the determination is a negative determination, acquire the second measurement value.

In the first aspect, the processor may be configured to, in a case where the determination is a negative determination, acquire the second measurement value that has been measured based on the second image.

In the first aspect, the processor may be configured to, in a case where the determination is a negative determination, measure the second measurement value based on the second image.

In the first aspect, the processor may be configured to, in a case where the determination is a negative determination: acquire, in a case where the second measurement value that has been measured based on the second image is present, the measured second measurement value; and measure the second measurement value based on the second image in a case where the measured second measurement value is not present.

In the first aspect, the processor may be configured to cause a display to display the second measurement value in different display forms in a case where the measured second measurement value is acquired and a case where the second measurement value is measured based on the second image.

In the first aspect, the processor may be configured to, in a case where the determination is a negative determination: acquire the second image; extract the second region of interest from the acquired second image; and measure the second measurement value based on the extracted second region of interest.

In the first aspect, the processor may be configured to cause a display to display the second measurement value acquired in the case where the determination is a negative determination in a display form different from the case where the second measurement value is included in the second document.

In the first aspect, the processor may be configured to: create a plot diagram of the acquired first measurement value and second measurement value; and cause a display to display the plot diagram.

In the first aspect, the first measurement value and the second measurement value may be at least one of a size or a signal value of the first region of interest and the second region of interest, respectively.

According to a second aspect of the present disclosure, there is provided an information processing method comprising: acquiring a first measurement value measured for a first region of interest included in a first image obtained by imaging a subject at a first point in time; acquiring a second document describing diagnosis content based on a second image obtained by imaging the subject at a second point in time; determining whether or not the second document includes a second measurement value of the same type as the first measurement value, which is measured for a second region of interest that is included in the second image and is the same as the first region of interest; and acquiring the second measurement value in a case where the determination is a negative determination.

According to a third aspect of the present disclosure, there is provided an information processing program for causing a computer to execute a process comprising: acquiring a first measurement value measured for a first region of interest included in a first image obtained by imaging a subject at a first point in time; acquiring a second document describing diagnosis content based on a second image obtained by imaging the subject at a second point in time; determining whether or not the second document includes a second measurement value of the same type as the first measurement value, which is measured for a second region of interest that is included in the second image and is the same as the first region of interest; and acquiring the second measurement value in a case where the determination is a negative determination.

With the information processing apparatus, the information processing method, and the information processing program according to the aspects of the present disclosure, it is possible to support comparative interpretation.

### BRIEF DESCRIPTION OF THE DRAWINGS

FIG. 1 is a diagram showing an example of a schematic configuration of an information processing system.

FIG. 2 is a diagram showing an example of a medical image.

FIG. 3 is a diagram showing an example of a medical image.

FIG. 4 is a block diagram showing an example of a hardware configuration of an information processing apparatus.

FIG. 5 is a block diagram showing an example of a functional configuration of the information processing apparatus.

FIG. 6 is a diagram showing an example of a screen displayed on a display.

FIG. 7 is a diagram showing an example of a screen displayed on a display.

FIG. 8 is a diagram showing an example of information registered in a report DB.

FIG. 9 is a diagram showing an example of a screen displayed on a display.

FIG. 10 is a diagram showing an example of a screen displayed on a display.

FIG. 11 is a diagram showing an example of a screen displayed on a display.

FIG. 12 is a diagram showing an example of a plot diagram.

FIG. 13 is a flowchart showing an example of information processing.

### DETAILED DESCRIPTION

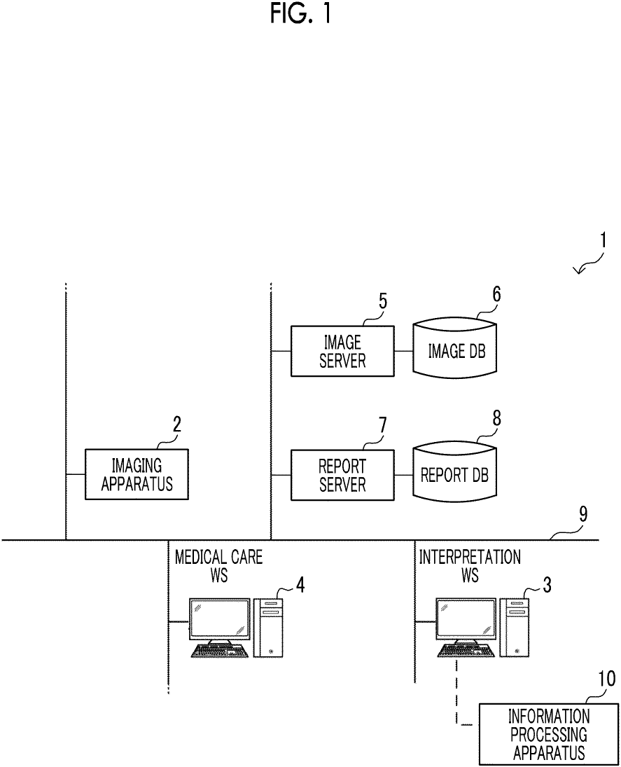

Hereinafter, an embodiment of the present disclosure will be described with reference to the drawings. First, a configuration of an information processing system 1 to which an information processing apparatus of the present disclosure is applied will be described. FIG. 1 is a diagram showing a schematic configuration of the information processing system 1. The information processing system 1 shown in FIG. 1 performs imaging of an examination target part of a subject and storing of a medical image acquired by the imaging based on an examination order from a doctor in a medical department using a known ordering system. In addition, the information processing system 1 performs an interpretation work of a medical image and creation of an interpretation report by a radiologist and viewing of the interpretation report by a doctor of a medical department that is a request source.

As shown in FIG. 1, the information processing system 1 includes an imaging apparatus 2, an interpretation work station (WS) 3 that is an interpretation terminal, a medical care WS 4, an image server 5, an image database (DB) 6, a report server 7, and a report DB 8. The imaging apparatus 2, the interpretation WS 3, the medical care WS 4, the image server 5, the image DB 6, the report server 7, and the report DB 8 are connected to each other via a wired or wireless network 9 in a communicable state.

Each apparatus is a computer on which an application program for causing each apparatus to function as a component of the information processing system 1 is installed. The application program may be recorded on, for example, a recording medium, such as a digital versatile disc read only memory (DVD-ROM) or a compact disc read only memory (CD-ROM), and distributed, and be installed on the computer from the recording medium. In addition, the application program may be stored in, for example, a storage apparatus of a server computer connected to the network 9 or in a network storage in a state in which it can be accessed from the outside, and be downloaded and installed on the computer in response to a request.

The imaging apparatus 2 is an apparatus (modality) that generates a medical image T showing a diagnosis target part of the subject by imaging the diagnosis target part. Examples of the imaging apparatus 2 include a simple X-ray imaging apparatus, a computed tomography (CT) apparatus, a magnetic resonance imaging (MRI) apparatus, a positron emission tomography (PET) apparatus, an ultrasound diagnostic apparatus, an endoscope, a fundus camera, and the like. The medical image generated by the imaging apparatus 2 is transmitted to the image server 5 and is saved in the image DB 6.

The interpretation WS 3 is a computer used by, for example, a healthcare professional such as a radiologist of a radiology department to interpret a medical image and to create an interpretation report, and encompasses an information processing apparatus 10 according to the present embodiment. In the interpretation WS 3, a viewing request for a medical image to the image server 5, various image processing for the medical image received from the image server 5, display of the medical image, and input reception of a sentence regarding the medical image are performed. In the interpretation WS 3, an analysis process for medical images, support for creating an interpretation report based on the analysis result, a registration request and a viewing request for the interpretation report to the report server 7, and display of the interpretation report received from the report server 7 are performed. The above processes are performed by the interpretation WS 3 executing software programs for respective processes.

The medical care WS 4 is a computer used by, for example, a healthcare professional such as a doctor in a medical department to observe a medical image in detail, view an interpretation report, create an electronic medical record, and the like, and is configured to include a processing apparatus, a display apparatus such as a display, and an input apparatus such as a keyboard and a mouse. In the medical care WS 4, a viewing request for the medical image to the image server 5, display of the medical image received from the image server 5, a viewing request for the interpretation report to the report server 7, and display of the interpretation report received from the report server 7 are performed. The above processes are performed by the medical care WS 4 executing software programs for respective processes.

The image server 5 is a general-purpose computer on which a software program that provides a function of a database management system (DBMS) is installed. The image server 5 is connected to the image DB 6. The connection form between the image server 5 and the image DB 6 is not particularly limited, and may be a form connected by a data bus, or a form connected to each other via a network such as a network attached storage (NAS) and a storage area network (SAN).

The image DB 6 is realized by, for example, a storage medium such as a hard disk drive (HDD), a solid state drive (SSD), and a flash memory. In the image DB 6, the medical image acquired by the imaging apparatus 2 and accessory information attached to the medical image are registered in association with each other.

The accessory information may include, for example, identification information such as an image identification (ID) for identifying a medical image, a tomographic ID assigned to each tomographic image included in the medical image, a subject ID for identifying a subject, and an examination ID for identifying an examination. In addition, the accessory information may include, for example, information related to imaging such as an imaging method, an imaging condition, and an imaging date and time related to imaging of a medical image. The “imaging method” and “imaging condition” are, for example, a type of the imaging apparatus 2, an imaging part, an imaging protocol, an imaging sequence, an imaging method, the presence or absence of use of a contrast medium, a slice thickness in tomographic imaging, and the like. In addition, the accessory information may include information related to the subject such as the name, date of birth, age, and gender of the subject. In addition, the accessory information may include information regarding the imaging purpose of the medical image.

In a case where the image server 5 receives a request to register a medical image from the imaging apparatus 2, the image server 5 prepares the medical image in a format for a database and registers the medical image in the image DB 6. In addition, in a case where the viewing request from the interpretation WS 3 and the medical care WS 4 is received, the image server 5 searches for a medical image registered in the image DB 6 and transmits the searched for medical image to the interpretation WS 3 and to the medical care WS 4 that are viewing request sources.

The report server 7 is a general-purpose computer on which a software program that provides a function of a database management system is installed. The report server 7 is connected to the report DB 8. The connection form between the report server 7 and the report DB 8 is not particularly limited, and may be a form connected by a data bus or a form connected via a network such as a NAS and a SAN.

The report DB 8 is realized by, for example, a storage medium such as an HDD, an SSD, and a flash memory. In the report DB 8, an interpretation report created in the interpretation WS 3 is registered.

Further, in a case where the report server 7 receives a request to register the interpretation report from the interpretation WS 3, the report server 7 prepares the interpretation report in a format for a database and registers the interpretation report in the report DB 8. Further, in a case where the report server 7 receives the viewing request for the interpretation report from the interpretation WS 3 and the medical care WS 4, the report server 7 searches for the interpretation report registered in the report DB 8, and transmits the searched for interpretation report to the interpretation WS 3 and to the medical care WS 4 that are viewing request sources.

In addition, although not described in the interpretation report, the report server 7 may store the result of analysis via computer aided detection/diagnosis (CAD) or the like at the time of creating the interpretation report (details will be described later).

The network 9 is, for example, a network such as a local area network (LAN) and a wide area network (WAN). The imaging apparatus 2, the interpretation WS 3, the medical care WS 4, the image server 5, the image DB 6, the report server 7, and the report DB 8 included in the information processing system 1 may be disposed in the same medical institution, or may be disposed in different medical institutions or the like. Further, the number of each apparatus of the imaging apparatus 2, the interpretation WS 3, the medical care WS 4, the image server 5, the image DB 6, the report server 7, and the report DB 8 is not limited to the number shown in FIG. 1, and each apparatus may be composed of a plurality of apparatuses having the same functions.

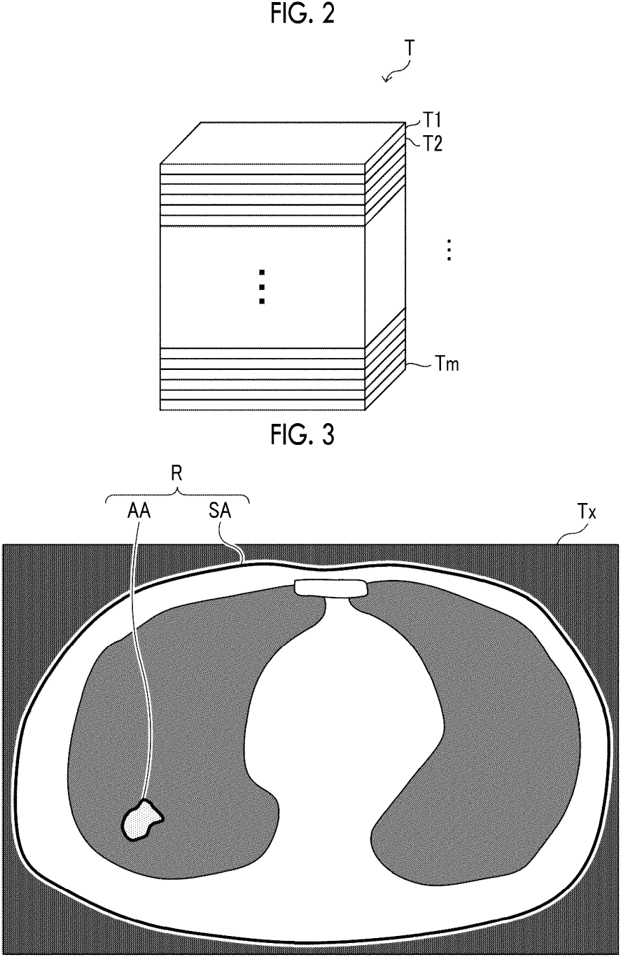

FIG. 2 is a diagram schematically showing an example of a medical image acquired by the imaging apparatus 2. The medical image T shown in FIG. 2 is, for example, a CT image consisting of a plurality of tomographic images T1 to Tm (m is 2 or more) representing tomographic planes from the head to the lumbar region of one subject (human body).

FIG. 3 is a diagram schematically showing an example of one tomographic image Tx out of the plurality of tomographic images T1 to Tm. The tomographic image Tx shown in FIG. 3 represents a tomographic plane including a lung. Each of the tomographic images T1 to Tm may include a region SA of a structure showing various organs and viscera of the human body (for example, lungs, livers, and the like), various tissues constituting various organs and viscera (for example, blood vessels, nerves, muscles, and the like), and the like. In addition, each tomographic image may include a region AA of an abnormal shadow showing lesions such as, for example, nodules, tumors, injuries, defects, and inflammation. In the tomographic image Tx shown in FIG. 3, the lung region is the region SA of the structure, and the nodule region is the region AA of the abnormal shadow. A single tomographic image may include regions SA of a plurality of structures and/or regions AA of a plurality of abnormal shadows. Hereinafter, at least one of the region SA of the structure or the region AA of the abnormal shadow is referred to as a “region of interest”.

Incidentally, a region of interest included in a medical image is detected and/or diagnosed by analyzing a medical image via CAD using a discriminator that has been trained by deep learning or the like. The creator of the interpretation report, such as a radiologist, creates the interpretation report with reference to the analysis result of the medical image via CAD. However, even though an analysis result of a medical image is obtained via CAD, the analysis result may not actually be described in the interpretation report, depending on the determination of the creator of the interpretation report. On the other hand, a lesion that was once determined not to be described in the interpretation report may be determined to be required to be described at the time of the re-examination due to deterioration over time or a change in the creator. In order to perform comparative interpretation in such a case, it takes time and effort to re-obtain the analysis result at a point in time not described in the interpretation report.

Therefore, the information processing apparatus 10 according to the present embodiment has a function of supporting comparative interpretation of medical images at a certain point in time even though an interpretation report at that point in time does not include the analysis result of the medical image. The information processing apparatus 10 will be described below. As described above, the information processing apparatus 10 is encompassed in the interpretation WS 3.

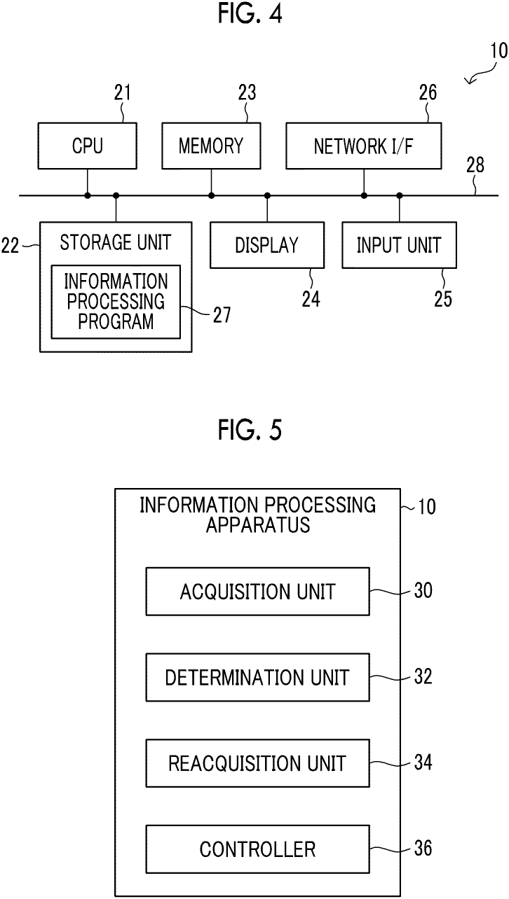

First, with reference to FIG. 4, an example of a hardware configuration of the information processing apparatus 10 according to the present embodiment will be described. As shown in FIG. 4, the information processing apparatus 10 includes a central processing unit (CPU) 21, a non-volatile storage unit 22, and a memory 23 as a temporary storage area. Further, the information processing apparatus 10 includes a display 24 such as a liquid crystal display, an input unit 25 such as a keyboard and a mouse, and a network interface (I/F) 26. The network I/F 26 is connected to the network 9 and performs wired or wireless communication. The CPU 21, the storage unit 22, the memory 23, the display 24, the input unit 25, and the network I/F 26 are connected to each other via a bus 28 such as a system bus and a control bus so that various types of information can be exchanged.

The storage unit 22 is realized by, for example, a storage medium such as an HDD, an SSD, and a flash memory. An information processing program 27 in the information processing apparatus 10 is stored in the storage unit 22. The CPU 21 reads out the information processing program 27 from the storage unit 22, loads the read-out program into the memory 23, and executes the loaded information processing program 27. The CPU 21 is an example of a processor of the present disclosure. As the information processing apparatus 10, for example, a personal computer, a server computer, a smartphone, a tablet terminal, a wearable terminal, or the like can be appropriately applied.

Next, with reference to FIG. 5, an example of a functional configuration of the information processing apparatus 10 according to the present embodiment will be described. As shown in FIG. 5, the information processing apparatus 10 includes an acquisition unit 30, a determination unit 32, a reacquisition unit 34, and a controller 36. The CPU 21 executes the information processing program 27, so that the CPU 21 functions as the acquisition unit 30, the determination unit 32, the reacquisition unit 34, and the controller 36.

In the following description, a medical image obtained by imaging a subject at a current point in time, that is, a medical image of a target for which a current interpretation report is created is referred to as a “current image”. In addition, a medical image obtained by imaging the same subject at a past point in time past the current point in time is referred to as a “past image”. The current point in time is an example of a first point in time of the present disclosure, and the current image is an example of a first image of the present disclosure. The past point in time is an example of a second point in time of the present disclosure, and the past image is an example of a second image of the present disclosure.

The acquisition unit 30 acquires findings information including at least measurement values measured for a region of interest included in the current image. Specifically, the acquisition unit 30 may acquire the findings information by acquiring a current image from the image server 5, extracting a region of interest from the current image, and performing image analysis on the region of interest.

The findings information includes at least measurement values measured for the region of interest, and may also include, for example, information indicating various findings such as a name (type), a property, a position, and an estimated disease name of the region of interest. The measurement value is a value that can be quantitatively measured from a medical image, and is, for example, at least one of a size or a signal value of a region of interest. The size is represented by, for example, a major axis, a minor axis, an area, a volume, or the like of a region of interest. The signal value is represented by, for example, a pixel value in a region of interest, a CT value in units of HU, or the like.

Examples of names (types) include the names of structures such as “lung” and “liver”, and the names of abnormal shadows such as “nodule”. The property mainly mean the features of abnormal shadows. For example, in the case of a lung nodule, findings indicating absorption values such as “solid type” and “frosted glass type”, margin shapes such as “clear/unclear”, “smooth/irregular”, “spicula”, “lobulation”, and “serration”, and an overall shape such as “round shape” and “irregular shape” can be mentioned. In addition, for example, there are findings regarding the relationship with surrounding tissues such as “pleural contact” and “pleural invagination”, and the presence or absence of contrast enhancement, washout, and the like.

The position means an anatomical position, a position in a medical image, and a relative positional relationship with other regions of interest such as “inside”, “margin”, and “periphery”. The anatomical position may be indicated by an organ name such as “lung” and “liver”, and may be expressed in terms of subdivided lungs such as “right lung”, “upper lobe”, and apical segment (“S1”). The estimated disease name is an evaluation result estimated based on the abnormal shadow, and, for example, the disease name such as “liver cirrhosis”, “cancer”, and “inflammation” and the evaluation result such as “negative/positive”, “benign/malignant”, and “mild/severe” regarding disease names and properties can be mentioned.

As a method for extracting the region of interest from the current image, a known method using a CAD technology, an artificial intelligence (AI) technology, or the like can be appropriately applied. For example, the acquisition unit 30 may extract the region of interest from the current image by receiving a medical image as an input and using a learning model such as a convolutional neural network (CNN) that has been trained to extract and output a region of interest included in the medical image. In addition, as a method of acquiring the findings information by image analysis, a known method using a CAD technology, an AI technology, or the like can be appropriately applied. For example, the acquisition unit 30 may acquire the measurement value by receiving the region of interest extracted from the medical image as an input and using a learning model such as a CNN that has been trained in advance to output the findings information of the region of interest.

Further, in a case where a plurality of regions of interest are extracted for the same subject, the acquisition unit 30 may acquire findings information for each of the plurality of regions of interest. The acquisition unit 30 may acquire findings information for each of a plurality of regions of interest included in one medical image, for example. Further, the acquisition unit 30 may acquire findings information for each of the plurality of regions of interest included in the plurality of tomographic images representing different tomographic planes, for example.

In addition, the acquisition unit 30 inquires of the report server 7 whether or not the past interpretation report describing diagnosis content based on the past image is registered in the report DB 8. In a case where a past interpretation report is registered in the report DB 8, the acquisition unit 30 acquires the past interpretation report. A past interpretation report describing diagnosis content based on the past image is an example of a second document of the present disclosure.

The determination unit 32 determines whether or not the past interpretation report acquired by the acquisition unit 30 includes a measurement value measured for a region of interest that is included in the past image and is the same as the region of interest included in the current image. As a method of determining whether or not the measurement value is included, for example, words related to measurement values (“major axis”, “minor axis”, “volume”, “cm”, “mm”, “cm³”, etc.) may be stored in the storage unit 22 in advance, and whether or not these words are included in the interpretation report may be determined. In addition, for example, a known named entity extraction method using a natural language processing model such as bidirectional encoder representations from transformers (BERT) may be appropriately applied.

Here, the region of interest included in the past image is the same as the region of interest included in the current image. Correlation between the regions of interest may be realized, for example, by aligning the past image and the current image. Hereinafter, the region of interest included in the current image is referred to as a “first region of interest”, and the region of interest that is included in the past image and is the same as the first region of interest is referred to as a “second region of interest”.

In addition, the measurement value measured for the second region of interest included in the past image is of the same type as the measurement value measured for the first region of interest included in the current image, and is, for example, at least one of the size or signal value of the second region of interest. Hereinafter, the measurement value for the first region of interest is referred to as a “first measurement value”, and a measurement value of the same type as the first measurement value for the second region of interest is referred to as a “second measurement value”. That is, the second measurement value is a past value of the first measurement value.

For example, it is assumed that the major axis (first measurement value) for the first region of interest included in the current image is acquired by the acquisition unit 30. In this case, the determination unit 32 determines whether or not the past image includes the same second region of interest as the first region of interest. In a case where the same second region of interest is included, the determination unit 32 further determines whether or not the major axis (second measurement value) for the second region of interest is measured and the major axis is included in the interpretation report.

In a case where the determination by the determination unit 32 is a negative determination, the reacquisition unit 34 acquires the second measurement value. For example, in a case where the determination by the determination unit 32 is a negative determination, the reacquisition unit 34 may acquire the second measurement value that has been measured based on the past image. For example, the reacquisition unit 34 may acquire the second measurement value stored in various storage media such as the storage unit 22, the report server 7, the report DB 8, the image server 5, the image DB 6, and other external devices.

Further, for example, in a case where the determination by the determination unit 32 is a negative determination, the reacquisition unit 34 may measure the second measurement value based on the past image. Specifically, in a case where the determination by the determination unit 32 is a negative determination, the reacquisition unit 34 may acquire the past image from the image server 5, extract the second region of interest from the acquired past image, and measure the second measurement value based on the extracted second region of interest. As a method of extracting the second region of interest from the past image and a method of acquiring the second measurement value, a known method using a CAD technology, an AI technology, or the like can be appropriately applied to the current image in the same manner as described above.

Further, for example, the reacquisition unit 34 may acquire the second measurement value by combining these. Specifically, in a case where the determination by the determination unit 32 is a negative determination, the reacquisition unit 34 may acquire the measured second measurement value from the various storage media in a case where the second measurement value that has been measured based on the past image is present in the various storage media, and measure the second measurement value based on the past image in a case where the measured second measurement value is not present in the various storage media.

The controller 36 controls the display 24 to display the second measurement value. Specifically, in a case where the determination by the determination unit 32 is an affirmative determination, that is, in a case where the second measurement value is included in the past interpretation report, the controller 36 extracts the second measurement value from the past interpretation report acquired by the acquisition unit 30 and causes the display 24 to display the second measurement value. On the other hand, in a case where the determination by the determination unit 32 is a negative determination, that is, in a case where the second measurement value is not included in the past interpretation report, the controller 36 causes the display 24 to display the second measurement value acquired by the reacquisition unit 34.

In addition, the controller 36 may cause the display 24 to display the second measurement value acquired by the reacquisition unit 34 in a case where the determination by the determination unit 32 is a negative determination in a display form different from that in the case where the second measurement value is included in the past interpretation report (that is, in the case where the determination by the determination unit 32 is an affirmative determination). For example, the controller 36 may change the underline, bold, oblique, size, color, and type of the character string representing the second measurement value, the background color of the character string, and the like, or may blink the character string.

Further, the controller 36 may cause the display 24 to display the second measurement value in different display forms in a case where the reacquisition unit 34 acquires the measured second measurement value from various storage media and a case where the reacquisition unit 34 measures the second measurement value based on the past image. This is because the acquisition source (past interpretation report, storage medium, or past image) of the second measurement value may be a material for determining whether or not the user describes the findings in the comment on findings.

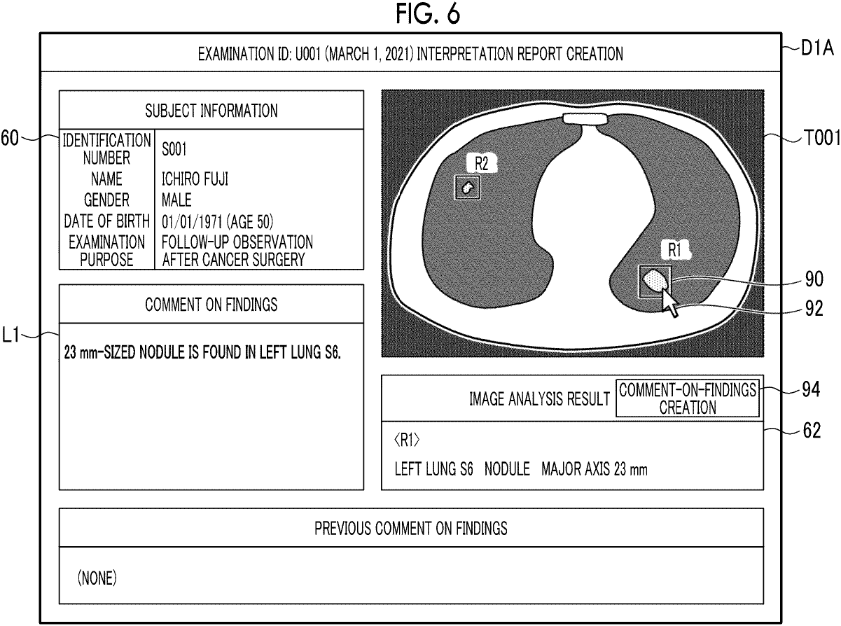

Hereinafter, examples of the information processing apparatus 10 according to the present embodiment will be described with reference to FIGS. 6 to 12.

In case of first examination First, a function of the information processing apparatus 10 in a case of creating an interpretation report in the first examination for a certain subject will be described with reference to FIGS. 6 to 8. FIG. 6 is an example of a screen D1A for creating an interpretation report, which is displayed on the display 24 by the controller 36. As shown in FIG. 6, the screen D1A includes subject information 60, a comment on findings L1, a medical image T001, and an image analysis result 62.

The medical image T001 is a medical image acquired from the image server 5 by the acquisition unit 30, and includes two regions of interest R1 and R2. Further, in the medical image T001, each of the regions of interest R1 and R2 is surrounded by a bounding box 90 and highlighted. The subject information 60 is information indicating a subject ID, a name, a gender, a date of birth, and an age of the subject, and an examination purpose, which are included in the accessory information of the medical image T001. The image analysis result 62 indicates findings information about the regions of interest R1 and/or R2 included in the medical image T001. In the case of the first examination, since the past interpretation report is not registered in the report DB 8, nothing is displayed in the field of “previous comment on findings”.

The user operates a mouse pointer 92 via the input unit 25 to select a region of interest for which the confirmation of the findings information is desired on the medical image T001. The acquisition unit 30 acquires findings information including at least the measurement value for the region of interest selected by the user. The controller 36 causes the display 24 to display the findings information acquired by the acquisition unit 30 as the image analysis result 62. The screen D1A shows a state in which the region of interest R1 is selected.

Next, in a case where the user confirms the image analysis result 62 (findings information) and determines that the comment on findings for the selected region of interest is to be described in the interpretation report, the user selects a comment-on-findings creation button 94. In a case where the comment-on-findings creation button 94 is selected, the controller 36 generates a comment on findings based on the findings information about the selected region of interest and causes the display 24 to display the comment on findings. The screen D1A shows a state in which the comment on findings L1 about the region of interest R1 is displayed.

Specifically, the controller 36 may generate a comment on findings including the findings information acquired by the acquisition unit 30 by machine learning. As a method of generating a comment on findings using machine learning, for example, a method using a recurrent neural network described in JP2019-153250A can be appropriately applied. Alternatively, for example, the controller 36 may generate a comment on findings by a known method of generating a comment on findings using a predetermined template.

In addition, the controller 36 may receive the correction by the user with respect to the comment on findings displayed on the display 24. In addition, the controller 36 may acquire not only comments on findings generated using machine learning but also comments on findings stored in advance in the report DB 8, the storage unit 22, or other external devices, and cause the display 24 to display the comments on findings. In addition, the controller 36 may receive the manual input of the comments on findings by the user.

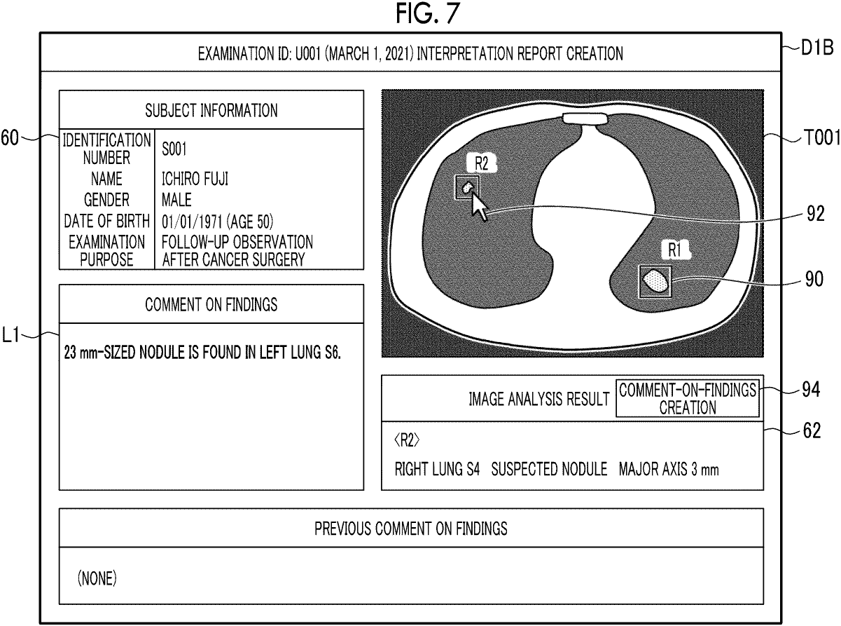

In addition, the user may select another region of interest on the medical image T001 in a case where he/she desires to confirm the findings information for the other region of interest. In a case where user selects another region of interest, the controller 36 controls the display 24 to display the findings information about the other region of interest. FIG. 7 is an example of a screen D1B in which the screen D1A (see FIG. 6) in a state in which the region of interest R1 is selected is transitioned to a state in which the region of interest R2 is selected.

Further, in a case where the user confirms the image analysis result 62 (findings information) and determines that the comment on findings for the selected region of interest does not have to be described in the interpretation report, the user does not have to select a comment-on-findings creation button 94. In this case, the controller 36 does not generate a comment on findings. On the screen D1B, the comment-on-findings creation button 94 is not selected for the region of interest R2, and the comment on findings L1 does not include the findings information about the region of interest R2.

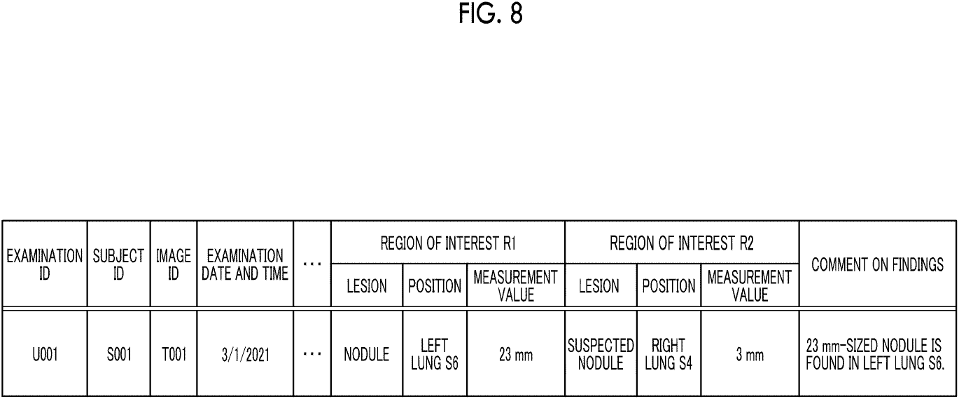

After the creation of the interpretation report is completed as described above, the controller 36 requests the image server 5 to register the medical image in the image DB 6, and requests the report server 7 to register the interpretation report including the findings information and the comment on findings in the report DB 8. FIG. 8 shows an example of contents registered in the report DB 8. As shown in FIG. 8, in the report DB 8, the findings information and the comment on findings L1 about the region of interest R1 described in the interpretation report and the findings information about the region of interest R2 not described in the interpretation report are registered. In addition, the examination ID, the subject ID, the image ID, other accessory information (not shown), and the like may be appropriately registered in the report DB 8.

In case where there is description in past interpretation report

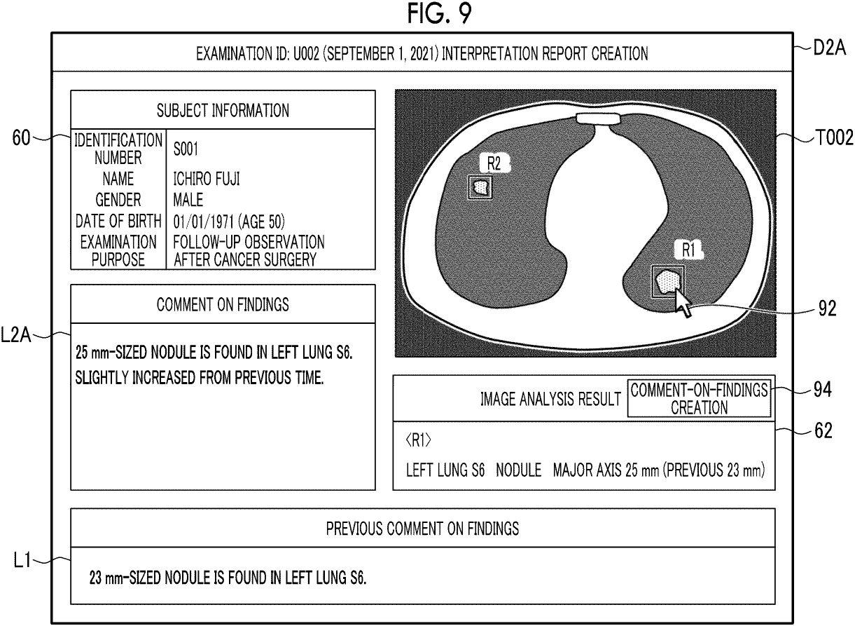

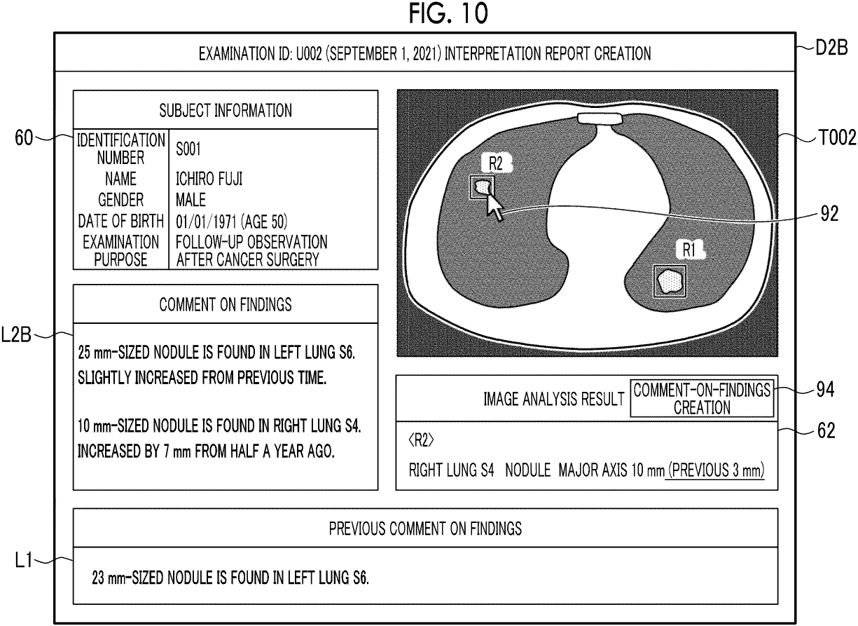

Next, a function of the information processing apparatus 10 in a case of creating an interpretation report in the second and subsequent examinations for a certain subject will be described with reference to FIG. 9. In the case of the second and subsequent examinations, unlike the case of the first examination, since the past interpretation reports are registered in the report DB 8, comparative interpretation can be performed while referring to the past interpretation reports.

Hereinafter, an example will be described in which the interpretation report describing the diagnosis content based on the medical image T001 created in FIGS. 6 to 8 is treated as a past interpretation report, and an interpretation report describing diagnosis content based on a medical image T002 is created on a screen D2A of FIG. 9. It should be noted that a part of the description overlapping with the description of the case of the first examination will be omitted.

FIG. 9 is an example of a screen D2A for creating an interpretation report, which is displayed on the display 24 by the controller 36. As shown in FIG. 9, the screen D2A includes the subject information 60, a comment on findings L2A, a medical image T002, the image analysis result 62, and the previous comment on findings L1.

The medical image T002 is a medical image acquired from the image server 5 by the acquisition unit 30, and includes two regions of interest R1 and R2. The previous comment on findings L1 is a comment on findings included in the past interpretation report acquired from the report server 7 by the acquisition unit 30.

The user operates a mouse pointer 92 via the input unit 25 to select a region of interest for which the confirmation of the findings information is desired on the medical image T002. The acquisition unit 30 acquires findings information including at least the measurement value (first measurement value) for the region of interest selected by the user. The determination unit 32 determines whether or not the past measurement value (second measurement value) for the region of interest selected by the user is included in the previous comment on findings L1 (past interpretation report).

The screen D2A shows a state in which the region of interest R1 is selected. In this case, the determination unit 32 determines that the previous comment on findings L1 includes the past measurement value for the region of interest R1. In a case where the determination unit 32 determines that the previous comment on findings L1 includes the past measurement value, the controller 36 extracts the past measurement value from the previous comment on findings L1 and causes the display 24 to display the past measurement value. On the screen D2A, a past measurement value of “(previous 23 mm)” is displayed on the image analysis result 62.

Next, in a case where the user confirms the image analysis result 62 (findings information) and the previous comment on findings L1 and determines that the comment on findings for the selected region of interest is to be described in the interpretation report, the user selects a comment-on-findings creation button 94. In a case where the comment-on-findings creation button 94 is selected, the controller 36 generates a comment on findings based on the findings information and the past measurement value about the selected region of interest and causes the display 24 to display the comment on findings. Specifically, the controller 36 may generate a comment on findings representing a comparison result with the past measurement value. The screen D2A shows a state in which the comment on findings L2A including the sentence representing the comparison result with the past measurement value of “slightly increased from the previous time” of the region of interest R1 is displayed.

In case where there is no description in past interpretation report

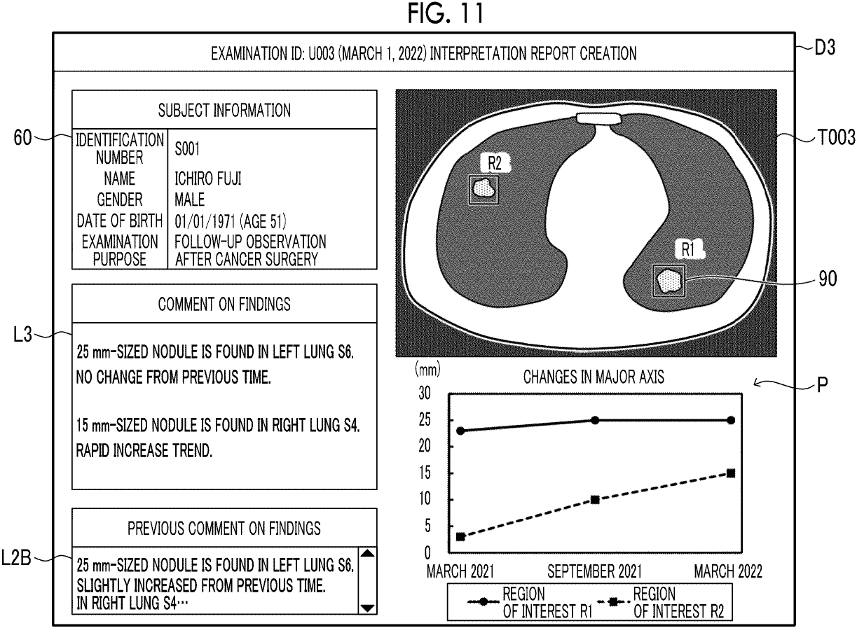

FIG. 10 is an example of a screen D2B in which the screen D2A (see FIG. 9) in a state in which the region of interest R1 is selected is transitioned to a state in which the region of interest R2 is selected. In this case, the determination unit 32 determines that the previous comment on findings L1 does not include the past measurement value for the region of interest R2.

In a case where the determination unit 32 determines that the past measurement value is not included in the previous comment on findings L1, the reacquisition unit 34 acquires the past measurement value that has been measured based on the medical image T001 from the findings information (see FIG. 8) registered in the report DB 8. The controller 36 causes the display 24 to display the past measurement value acquired by the reacquisition unit 34. On the screen D2B, a past measurement value of “(previous 3 mm)” is displayed on the image analysis result 62.

In addition, the “(previous 3 mm)” of the image analysis result 62 on the screen D2B is underlined, and the “(previous 23 mm)” of the image analysis result 62 on the screen D2A is not underlined. In this way, the controller 36 may change whether to underline the past measurement values (“(previous 23 mm)” and “(previous 3 mm)”) of the image analysis result 62 depending on whether or not the second measurement values are included in the past interpretation reports.

In a case where the comment-on-findings creation button 94 is selected by the user in a state in which the region of interest R2 is selected, the controller 36 adds a comment on findings based on the findings information and the past measurement value about the selected region of interest R2 and causes the display 24 to display the comment on findings. The screen D2B shows a state in which a comment on findings L2B including the sentence about the region of interest R1 and the sentence about the region of interest R2 is displayed.

In case of displaying plot diagram of measurement values

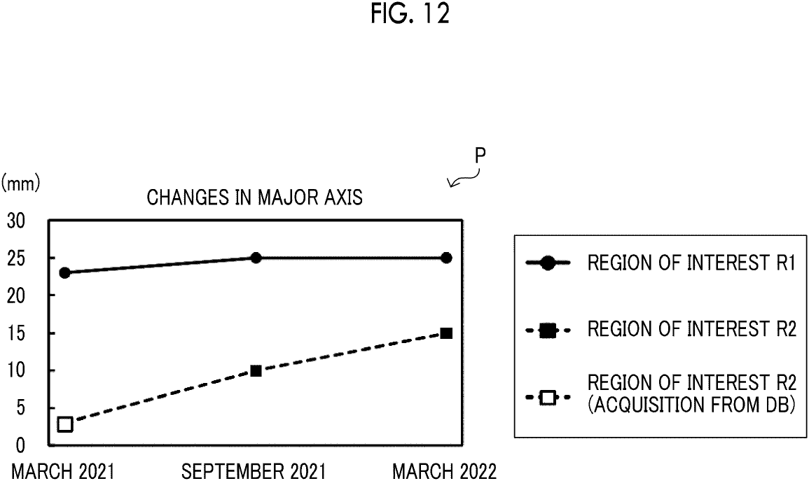

The controller 36 may create a plot diagram of the first measurement value acquired by the acquisition unit 30 and the second measurement value acquired by the reacquisition unit 34 and control the display 24 to display the plot diagram. FIG. 11 shows an example of a screen D3 in which a plot diagram is displayed on a screen for creating an interpretation report, which is displayed on the display 24 by the controller 36. Hereinafter, the screen D3 is a screen for treating the interpretation report describing the diagnosis content based on the medical image T002 created in FIGS. 9 and 10 as a past interpretation report, and creating an interpretation report describing diagnosis content based on a medical image T003. As shown in FIG. 11, the screen D3 includes the subject information 60, a comment on findings L3, the medical image T003, the previous comment on findings L2B, and a plot diagram P.

Specifically, the controller 36 may create a plot diagram P showing a line graph with the measurement value as the vertical axis and the point in time of measurement (time axis) of the measurement value as the horizontal axis. By presenting the changes in the measurement values as the plot diagram P in this way, it is possible to easily grasp the changes over time in measurement values.

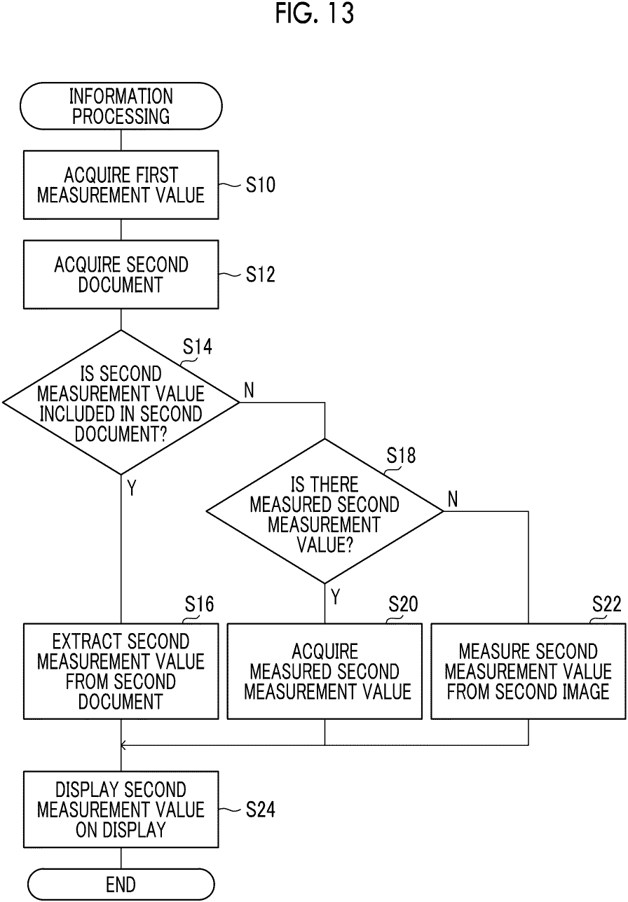

In addition, as shown in FIG. 12, the controller 36 may change display form of plots between the second measurement value acquired by the reacquisition unit 34 in a case where the determination by the determination unit 32 is a negative determination and the case where the second measurement value is included in the past interpretation report. FIG. 12 is a modification example of the plot diagram P of FIG. 11, and a plot as of March 2021 acquired from the report DB 8 by the reacquisition unit 34 is displayed in a different form from other plots extracted from the comment on findings by the controller 36.

Further, the controller 36 may change the display form of plots in a case where the reacquisition unit 34 acquires the measured second measurement value from various storage media and a case where the reacquisition unit 34 measures the second measurement value based on the past image.

Next, with reference to FIG. 13, operations of the information processing apparatus 10 according to the present embodiment will be described. In the information processing apparatus 10, as the CPU 21 executes the information processing program 27, information processing shown in FIG. 13 is executed. The information processing is executed, for example, in a case where the user gives an instruction to start execution via the input unit 25.

In Step S10, the acquisition unit 30 acquires a first measurement value measured for the first region of interest included in the first image (current image) obtained by imaging the subject at the first point in time (current point in time). In Step S12, the acquisition unit 30 acquires a second document (past interpretation report) describing the diagnosis content based on the second image (past image) obtained by imaging the subject at the second point in time (past point in time).

In Step S14, the determination unit 32 determines whether or not the second document acquired in Step S12 includes a second measurement value of the same type as the first measurement value acquired in Step S10, which is included in the second image and is measured for the second region of interest that is the same as the first region of interest. In a case where an affirmative determination is made in Step S14, the process proceeds to Step S16. In Step S16, the controller 36 extracts the second measurement value from the second document acquired in Step S12.

On the other hand, in a case where a negative determination is made in Step S14, the process proceeds to Step S18. In Step S18, the reacquisition unit 34 determines whether or not the second measurement value that has been measured based on the second image is present in various storage media (the storage unit 22, the report server 7, the report DB 8, the image server 5, the image DB 6, other external devices, and the like). In a case where an affirmative determination is made in Step S18, the process proceeds to Step S20. In Step S20, the reacquisition unit 34 acquires the measured second measurement value stored in various storage media.

On the other hand, in a case where a negative determination is made in Step S18, the process proceeds to Step S22. In Step S22, the reacquisition unit 34 acquires the second image from the image server 5 and measures the second measurement value based on the second image. In Step S24, the controller 36 controls the display 24 to display the second measurement value acquired in any one of Step S16, Step S18, or Step S22, and ends the present information processing.

As described above, the information processing apparatus 10 according to one aspect of the present disclosure comprises at least one processor, and the processor acquires a first measurement value measured for a first region of interest included in a first image obtained by imaging a subject at a first point in time, acquires a second document describing diagnosis content based on a second image obtained by imaging the subject at a second point in time, determines whether or not the second document includes a second measurement value of the same type as the first measurement value, which is measured for a second region of interest that is included in the second image and is the same as the first region of interest, and in a case where the determination is a negative determination, acquires the second measurement value.

That is, with the information processing apparatus 10 according to the present embodiment, it is possible to acquire the measurement value at the past point in time even though the measurement value for the region of interest of the target currently being interpreted is not included in the past interpretation report. Therefore, for example, even in a case where a lesion that was once determined not to be described in the interpretation report is determined to be required to be described at the time of the re-examination due to deterioration over time or a change in the creator, it is possible to acquire the measurement value at the past point in time. That is, with the information processing apparatus 10 according to the present embodiment, it is possible to support comparative interpretation.

In addition, in the above-described embodiment, a form in which the acquisition unit 30 acquires findings information including at least a measurement value by performing image analysis on a medical image has been described, but the present disclosure is not limited thereto. For example, the acquisition unit 30 may acquire findings information stored in advance in the storage unit 22, the image server 5, the image DB 6, the report server 7, the report DB 8, and other external devices. Alternatively, for example, the acquisition unit 30 may acquire findings information manually input by the user via the input unit 25.

Further, in the above-described embodiment, a form has been described assuming a situation in which an interpretation report is created in the interpretation WS 3, but the present disclosure is not limited thereto. For example, the information processing apparatus 10 may be applied to a situation in which two interpretation reports are compared and viewed in the interpretation WS 3 and/or the medical care WS 4.

Further, in the above-described embodiment, a form assuming an interpretation report for medical images has been described, but the present disclosure is not limited thereto. The information processing apparatus 10 of the present disclosure can be applied to various documents describing diagnosis content based on an image obtained by imaging a subject. For example, the information processing apparatus 10 may be applied to a document describing diagnosis content based on an image acquired using an apparatus, a building, a pipe, a welded portion, or the like as a subject in a non-destructive examination such as a radiation transmission examination and an ultrasonic flaw detection examination.

In the above embodiment, for example, as hardware structures of processing units that execute various kinds of processing, such as the acquisition unit 30, the determination unit 32, the reacquisition unit 34, and the controller 36, various processors shown below can be used. As described above, the various processors include a programmable logic device (PLD) as a processor of which the circuit configuration can be changed after manufacture, such as a field programmable gate array (FPGA), a dedicated electrical circuit as a processor having a dedicated circuit configuration for executing specific processing such as an application specific integrated circuit (ASIC), and the like, in addition to the CPU as a general-purpose processor that functions as various processing units by executing software (program).

One processing unit may be configured by one of the various processors, or may be configured by a combination of the same or different kinds of two or more processors (for example, a combination of a plurality of FPGAs or a combination of the CPU and the FPGA). In addition, a plurality of processing units may be configured by one processor.

As an example in which a plurality of processing units are configured by one processor, first, there is a form in which one processor is configured by a combination of one or more CPUs and software as typified by a computer, such as a client or a server, and this processor functions as a plurality of processing units. Second, as represented by a system on chip (SoC) or the like, there is a form of using a processor for realizing the function of the entire system including a plurality of processing units with one integrated circuit (IC) chip. In this way, various processing units are configured by one or more of the above-described various processors as hardware structures.

Furthermore, as the hardware structure of the various processors, more specifically, an electrical circuit (circuitry) in which circuit elements such as semiconductor elements are combined can be used.

In the above embodiment, the information processing program 27 is described as being stored (installed) in the storage unit 22 in advance; however, the present disclosure is not limited thereto. The information processing program 27 may be provided in a form recorded in a recording medium such as a compact disc read only memory (CD-ROM), a digital versatile disc read only memory (DVD-ROM), and a universal serial bus (USB) memory. In addition, the information processing program 27 may be downloaded from an external device via a network. Further, the technique of the present disclosure extends to a storage medium for storing the information processing program non-transitorily in addition to the information processing program.

The technique of the present disclosure can be appropriately combined with the above-described embodiment and examples. The described contents and illustrated contents shown above are detailed descriptions of the parts related to the technique of the present disclosure, and are merely an example of the technique of the present disclosure. For example, the above description of the configuration, function, operation, and effect is an example of the configuration, function, operation, and effect of the parts according to the technique of the present disclosure. Therefore, needless to say, unnecessary parts may be deleted, new elements may be added, or replacements may be made to the described contents and illustrated contents shown above within a range that does not deviate from the gist of the technique of the present disclosure.

What is claimed is:

1. An information processing apparatus comprising at least one processor, wherein the at least one processor is configured to:
acquire a first measurement value measured for a first region of interest included in a first image obtained by imaging a subject at a first point in time;
acquire a second document describing diagnosis content based on a second image obtained by imaging the subject at a second point in time;
determine whether or not the second document includes a second measurement value of the same type as the first measurement value, which is measured for a second region of interest included in the second image, the second region of interest being the same as the first region of interest; and
in a case where the determination is a negative determination, acquire the second measurement value.

2. The information processing apparatus according to claim 1, wherein the at least one processor is configured to, in a case where the determination is a negative determination, acquire the second measurement value that has been measured based on the second image.

3. The information processing apparatus according to claim 1, wherein the at least one processor is configured to, in a case where the determination is a negative determination, measure the second measurement value based on the second image.

4. The information processing apparatus according to claim 1, wherein the at least one processor is configured to, in a case where the determination is a negative determination:
acquire, in a case where the second measurement value that has been measured based on the second image is present, the measured second measurement value; and
measure the second measurement value based on the second image in a case where the measured second measurement value is not present.

5. The information processing apparatus according to claim 4, wherein the at least one processor is configured to cause a display to display the second measurement value in different display forms in a case where the measured second measurement value is acquired and a case where the second measurement value is measured based on the second image.

6. The information processing apparatus according to claim 3, wherein the at least one processor is configured to, in a case where the determination is a negative determination:
acquire the second image;
extract the second region of interest from the acquired second image; and
measure the second measurement value based on the extracted second region of interest.

7. The information processing apparatus according to claim 1, wherein the at least one processor is configured to cause a display to display the second measurement value acquired in the case where the determination is a negative determination in a display form different from the case where the second measurement value is included in the second document.

8. The information processing apparatus according to claim 1, wherein the at least one processor is configured to:
create a plot diagram of the acquired first measurement value and second measurement value; and
cause a display to display the plot diagram.

9. The information processing apparatus according to claim 1, wherein the first measurement value and the second measurement value are at least one of a size or a signal value of the first region of interest and the second region of interest, respectively.

10. An information processing method comprising:
acquiring a first measurement value measured for a first region of interest included in a first image obtained by imaging a subject at a first point in time;
acquiring a second document describing diagnosis content based on a second image obtained by imaging the subject at a second point in time;
determining whether or not the second document includes a second measurement value of the same type as the first measurement value, which is measured for a second region of interest included in the second image, the second region of interest being the same as the first region of interest; and
acquiring the second measurement value in a case where the determination is a negative determination.

11. A non-transitory computer-readable storage medium storing an information processing program for causing a computer to execute a process comprising:
acquiring a first measurement value measured for a first region of interest included in a first image obtained by imaging a subject at a first point in time;
acquiring a second document describing diagnosis content based on a second image obtained by imaging the subject at a second point in time;
determining whether or not the second document includes a second measurement value of the same type as the first measurement value, which is measured for a second region of interest included in the second image, the second region of interest being the same as the first region of interest; and
acquiring the second measurement value in a case where the determination is a negative determination.
